(19) United States
(12) Patent Application Publication
(10) Pub. No.: US 2007/0282257 A1
(43) Pub. Date: Dec. 6, 2007

(54) MYOCARDIAL INJECTOR WITH BALLOON ABUTMENT

(76) Inventor: Richard A. Schatz, Rancho Santa Fe, CA (US)

Correspondence Address: NYDEGGER & ASSOCIATES, 348 OLIVE STREET, SAN DIEGO, CA, 92103, US

(21) Appl. No.: 11/558,824

(22) Filed: Nov. 10, 2006

Related U.S. Application Data
(63) Continuation-in-part of application No. 11/422,307, filed on Jun. 5, 2006.

(51) Int. Cl. A61M 29/00

(52) U.S. Cl. 604/96.01

(57) ABSTRACT

A device is provided for performing intra myocardial injections. The device comprises a needle, a catheter formed with a lumen, and an inflatable abutment member. The abutment member is movable between a first configuration, wherein it is substantially tube-like and is deflated, and a second configuration, wherein it inflates beyond the distal end of the catheter and extends radially. Before the needle is advanced to perform an injection, the abutment member is moved to its second configuration to prevent contact between the catheter and the myocardial tissue during the injection.

This application is a continuation-in-part of application Ser. No. 11/422,307 filed Jun. 5, 2006 which is currently pending. The contents of application Ser. No. 11/422,307 are incorporated herein by reference.

### FIELD OF THE INVENTION

The present invention pertains generally to devices and methods for delivering medicaments to a patient. More specifically, the present invention pertains to devices and methods for performing intra myocardial injections. The present invention is particularly, but not exclusively, useful as a device and method for advancing a needle from a catheter to perform an intra myocardial injection while protecting the myocardial tissue from contact with the catheter.

### BACKGROUND OF THE INVENTION

Intravascular catheters are used in a wide variety of medical procedures by inserting the catheter into the vascular system of the patient at an easily accessible location. Thereafter, the tip of the catheter is advanced through the vasculature to a desired target site. In this manner, virtually any target site in the patient's vascular system may be remotely accessed. Of particular interest here are those medical procedures that require the use of injection catheters to inject therapeutic or diagnostic agents into various target tissues within the human body. When so used, an advantage of injection catheters is that the target tissue may be accessed by minimally invasive surgical techniques.

In many applications the target tissue is within a wall of an organ, such as the heart. For instance, therapeutic or diagnostic agents such as genes, proteins, stem cells, or drugs may be injected directly into the heart. When the target tissue is within the wall of an organ, however, it is often desirable to inject the therapeutic or diagnostic agent into the tissue at a specific site in the organ wall. In these applications, if the needle of the injection catheter inadvertently passes through the wall, the therapeutic or diagnostic agents that are dispensed from the distal end of the needle will not be effectively delivered to the target tissue. Further, because the injection procedure often requires the thrust of a needle in the distal direction, the required motion can cause the catheter itself to contact and perforate or otherwise injure the wall of the organ, resulting in a life threatening situation.

In light of the above, it is an object of the present invention to provide a device and method that protects the myocardial wall from injury and perforation during an intra myocardial injection. Another object of the invention is to provide a device and method for controlling the depth of an intra myocardial injection. Still another object of the invention is to provide a device and method for performing an intra myocardial injection from a catheter in which a removable barrier prevents contact between the catheter and the myocardial tissue and allows the physician to advance the needle with confidence and without fear of perforating the myocardial tissue with the catheter. Yet another object of the present invention is to provide a device and method for performing intra myocardial injections which is easy to implement, simple to perform, and cost effective.

### SUMMARY OF THE INVENTION

In accordance with the present invention, an injector is provided to perform intra myocardial injections while preventing unnecessary trauma to the myocardial tissue. Structurally, the injector includes a catheter having a proximal end and a distal end. Further, the catheter is formed with a lumen that extends from the proximal end to the distal end and defines a longitudinal axis. The injector also includes a needle that is mounted within the lumen of the catheter for axial movement therein.

An important aspect of the injector of the present invention is an inflatable abutment member that is mounted to the catheter. For purposes of the present invention, this inflatable abutment member is tube-shaped and is movable between a first configuration and a second configuration. In the first configuration, the abutment member is deflated and is held alongside the catheter. In the second configuration, the abutment member is inflated and extends radially from and beyond the distal end of the catheter. Further, in the second configuration, the abutment member maintains a central void for passage of the needle tip through the central void.

Also, the inflatable abutment member includes a port that is in fluid communication with a fluid source, such as a reservoir of saline with iodinated contrast. Specifically, the injector includes a tube that connects the port on the abutment member with the saline reservoir.

For the operation of the present invention, saline with iodinated contrast is transferred between the saline reservoir and the abutment member to move the abutment member between the first configuration and the second configuration. In the first configuration, as mentioned above, the abutment member is substantially tube-like. In the second configuration, the abutment member extends radially from and beyond the distal end of the catheter.

In order to perform an intra myocardial injection, the injector is placed at a desired position near or adjacent myocardial tissue. During this placement, the abutment member is in its first configuration. Thereafter, saline or contrast or a mixture of both is transferred from the saline reservoir to the abutment member to expand the abutment member in the distal direction through the distal end of the catheter until the abutment member reaches the second configuration. In the second configuration, the distal portion of the abutment member flares radially. Because the abutment member extends distally from the catheter, the abutment member provides a barrier between the catheter and the myocardial tissue. Therefore, when the needle is advanced (through the central void in the abutment member) and the needle tip penetrates the myocardial tissue to perform the injection, the abutment member limits or minimizes contact between the catheter and the myocardial tissue, preventing unwanted advancement of the catheter. As a result, the surgeon may confidently advance the needle without risk of damaging the myocardial tissue with the catheter.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of the intra myocardial injector in accordance with the present invention;

FIG. 2 is a perspective view of an embodiment of the intra myocardial injector of FIG. 1 shown with the abutment member in its second configuration in accordance with the present invention;

FIG. 3A is a cross sectional view of the injector of FIG. 2 shown with the abutment member in its first configuration in accordance with the present invention;

FIG. 3B is a cross sectional view of the injector of FIG. 2 shown with the abutment member in its second configuration in accordance with the present invention;

FIG. 3C is a cross sectional view of the injector of FIG. 2 shown with the abutment member in its second configuration and with the needle advanced and penetrating myocardial tissue in accordance with the present invention;

FIG. 4 is a perspective view of another embodiment of the intra myocardial injector of FIG. 1 shown with the abutment member in its extended (second) configuration in accordance with the present invention;

FIG. 5A is a cross sectional view of an embodiment of the injector of FIG. 4 shown with the abutment member in its first configuration in accordance with the present invention;

FIG. 5B is a cross sectional view of the injector shown in FIG. 5A shown with the abutment member in its second configuration in accordance with the present invention;

FIG. 5C is a cross sectional view of the injector shown in FIG. 5B with the needle advanced and penetrating myocardial tissue in accordance with the present invention;

FIG. 6A is a cross sectional view of an alternate embodiment of the injector of FIG. 4 shown with the abutment member in its first configuration in accordance with the present invention;

FIG. 6B is a cross sectional view of the injector shown in FIG. 6A shown with the abutment member in its second configuration in accordance with the present invention; and

FIG. 6C is a cross sectional view of the injector shown in FIG. 6B with the needle advanced and penetrating myocardial tissue in accordance with the present invention.

### DESCRIPTION OF THE PREFERRED EMBODIMENTS

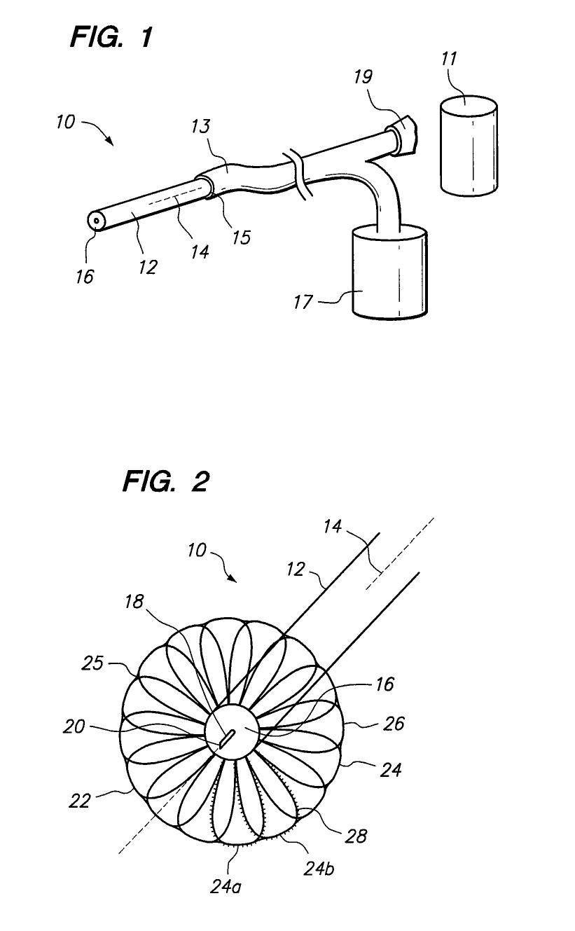

Referring initially to FIG. 1, an intra myocardial injector in accordance with the present invention is shown, and is generally designated 10. As shown in FIG. 1, the injector 10 includes a catheter 12 that extends along an axis 14 from a proximal end 15 to a distal end 16. As is shown, the proximal end 15 of the catheter 12 is connected to tubing 13. For purposes of the present invention, the tubing 13 is in fluid communication with a vessel 17 for holding medicament or other fluid for medical treatment. As is further shown, the tubing 13 also includes a port 19 that provides access for manipulation of internal components of the catheter 12. For instance, the port 19 may connect to a fluid reservoir 11.

Referring now to FIG. 2, the injector 10 is shown to include a needle 18 that terminates at a needle tip 20. As shown in FIG. 2, the needle 18 has been advanced so that the needle tip 20 extends beyond the distal end 16 of the catheter 12. Further, the injector 10 is shown to include an abutment member 22. As shown, the abutment member 22 is formed from a plurality of loops 24 of wire 26. Specifically, the abutment member 22 is formed with overlapping loops 24 that are biased to radially extend from the axis 14. Further, the abutment member 22 may include a webbing 25 that interconnects adjacent loops 24. Such a webbing 25 can comprise a high-friction material. For the present invention, the abutment member 22 may include engagement elements 28 such as tines that extend from exemplary loops 24a, 24b, or a high friction surface.

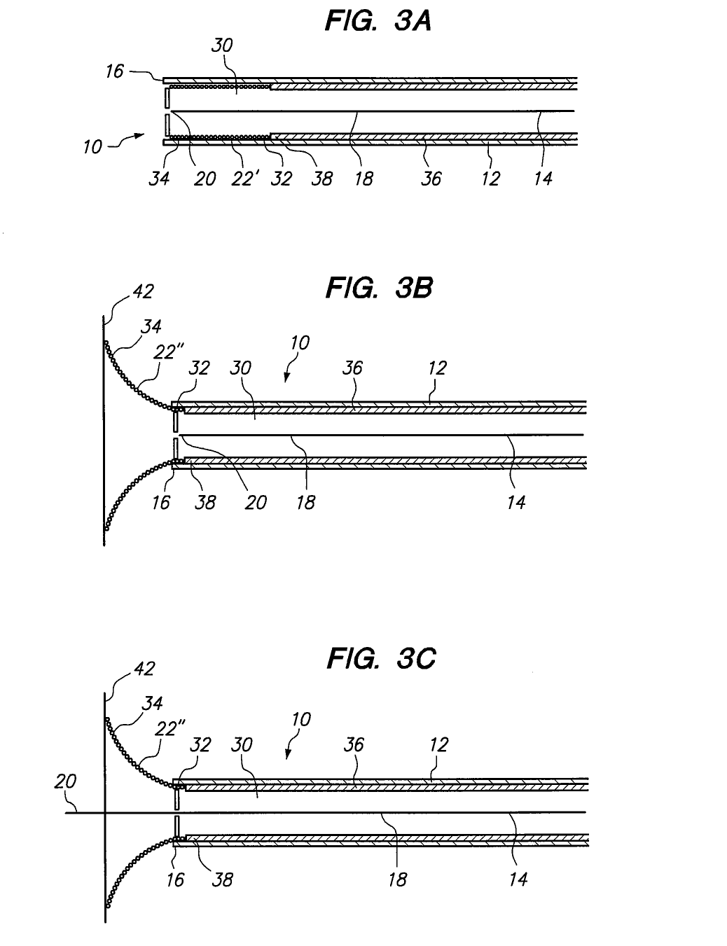

Turning now to FIG. 3A, the internal features of the injector 10 may be understood. As shown in FIG. 3A, the catheter 12 forms a lumen 30 that extends along the axis 14 to the distal end 16. Unlike in FIG. 2, the abutment member 22′ is positioned completely within the lumen 30, i.e., in its first configuration. As shown, when in its first configuration, the abutment member 22′ is substantially tube-like. Specifically, the abutment member 22′ includes a proximal portion 32 and a distal portion 34 that are substantially cylindrical.

In FIG. 3A, it is further shown that the injector 10 includes a pusher rod 36 that includes a distal end 38. As shown, the pusher rod 36 is received within the lumen 30 and is axially movable with respect to the catheter 12. For purposes of the present invention, the distal end 38 of the pusher rod 36 engages the proximal portion 32 of the abutment member 22′ to cause movement of the abutment member 22′. In certain embodiments, the rod 36 and the abutment member 22 may be a single piece.

Still referring to FIG. 3A, the needle 18 is shown positioned entirely within the lumen 30, with the end 16 of the catheter 12 distal of the needle tip 20. Structurally, the needle 18 may be mounted to a needle hub (not shown) for movement with respect to the lumen 30 of the catheter 12.

Referring now to FIG. 3B, the abutment member 22″ is shown in its second configuration. Specifically, as shown, the distal portion 34 of the abutment member 22″ is shown extended from the distal end 16 of the catheter 12. Because it is radially biased, the distal portion 34 of the abutment member 22″ flares radially when extended beyond the distal end 16 of the catheter 12 and takes a fan shape. As shown in FIG. 3B, the needle 18 is still retracted within the catheter 12.

Referring now to FIG. 3C, it can be seen that the needle 18 has been advanced to extend the needle tip 20 beyond the distal end 16 of the catheter 12 and the abutment member 22″. Preferably, the needle tip 20 extends between 3 and 7 millimeters beyond the distal portion 34 of the abutment member 22″.

Referring now to FIGS. 3A-3C collectively, the operation of the present invention may be understood. Initially, the injector 10 is configured as in FIG. 3A with the abutment member 22′ in its first configuration. When an intra myocardial injection is desired, the injector 10 is placed adjacent myocardial tissue 42. Next, as shown in FIG. 3B, the pusher rod 36 is advanced and the distal end 38 of the rod 36 forces the abutment member 22 in the distal direction. The abutment member 22 is moved distally until it reaches its second configuration. In the second configuration, the distal portion 34 of the abutment member 22″ flares radially and provides a barrier between the distal end 16 of the catheter 12 and the myocardial tissue 42. When the injector 10 is moved toward the myocardial tissue 42, the abutment member 22 abuts the tissue 42 and prevents further movement of the catheter 12 in the distal direction. Further, the engagement elements 28 (shown in FIG. 2) on the abutment member 22 may pierce or contact and engage the myocardial tissue 42 to anchor the injector 10 in position.

With the abutment member 22″ in its second configuration, the needle 18 may be advanced to extend the needle tip 20 as shown in FIG. 3C. Advancement of the needle 18 may be accomplished by pushing the needle hub (not shown) relative to the catheter 12, or otherwise pushing the needle 18 relative to the catheter 12 as known in the art. As a result of its advancement, the needle tip 20 extends beyond the distal end 16 of the catheter 12 and beyond the distal portion 34 of the abutment member 22″ to penetrate the myocardial tissue 42 to an approximate depth of between 3 and 7 millimeters. When the myocardial tissue 42 has been penetrated by the needle tip 20 at the appropriate depth, a medicament or other treatment fluid may be injected through the needle tip 20 as is well known in the art.

After the injection has been performed, the needle 18 is retracted within the lumen 30, as illustrated in FIG. 3B. Then the abutment member 22″ is withdrawn into the lumen 30 by the pusher rod 36, as illustrated in FIG. 3A. Thereafter, the injector 10 is removed from the patient's vasculature.

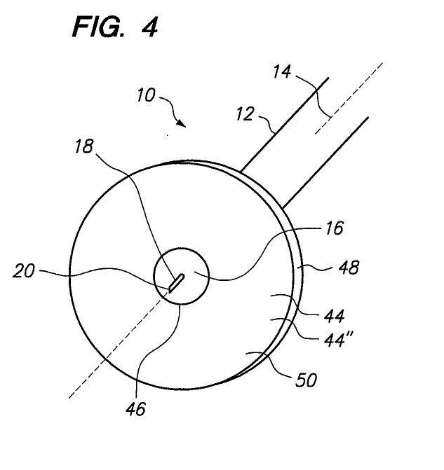

Referring now to FIG. 4, the injector 10 is again shown to include a needle 18 that terminates at a needle tip 20. Similar to the injector 10 shown in FIG. 2, the needle 18 has been advanced so that the needle tip 20 extends beyond the distal end 16 of the catheter 12. Further, the injector 10 is shown to include an inflatable abutment member 44. As shown, the abutment member 44 is formed from a flexible bladder such as a balloon. For purposes of the present invention, the abutment member 44 may be round, flat, elliptical, or any desired shape. As shown, the abutment member 44 has a proximal portion 46 mounted to the catheter 12. For the present invention, the abutment member 44 may include a high friction surface 50.

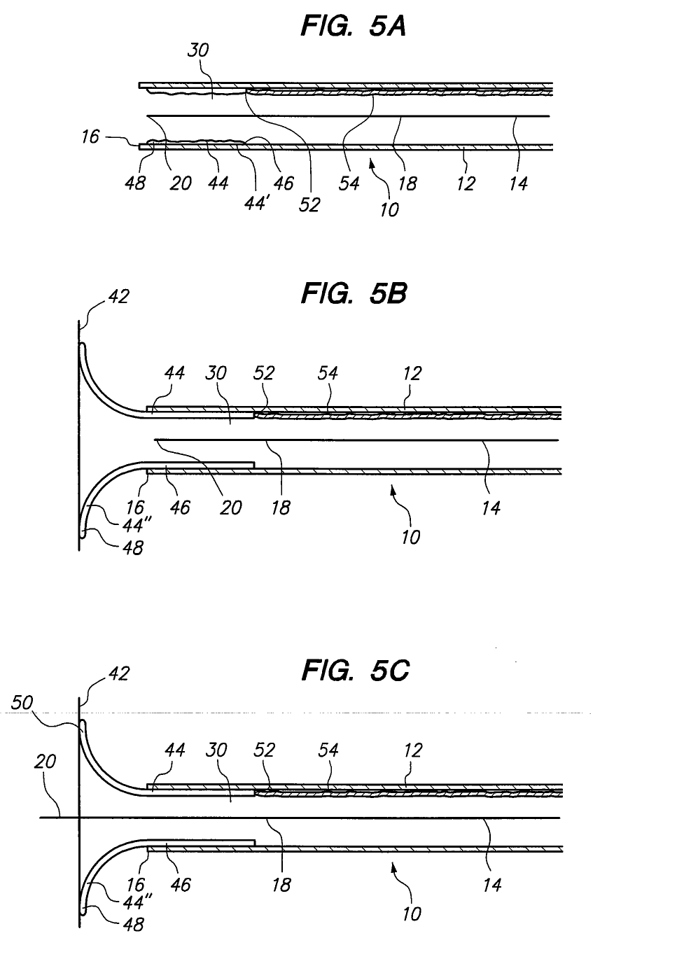

Turning now to FIG. 5A, the internal features of the injector 10 may be understood. As shown in FIG. 5A, the catheter 12 forms a lumen 30 that extends along the axis 14 to the distal end 16. Unlike in FIG. 4, the abutment member 44′ is deflated and positioned completely within the lumen 30, i.e., in its first configuration. As shown, when in its first configuration, the abutment member 44′ is substantially tube-like. Specifically, the proximal portion 46 and the distal portion 48 of the abutment member 44′ are substantially cylindrical. For purposes of the present invention, the proximal portion 46 of the abutment member 44 may be mounted to the catheter 12.

In FIG. 5A, it is further shown that the abutment member 44 includes a port 52 that is in fluid communication with a tube 54. For purposes of the present invention, the tube 54 is connected to the fluid reservoir 11 (shown in FIG. 1) to transfer fluid between the reservoir 11 and the abutment member 44′ to cause movement of the abutment member 44′. In certain embodiments, the abutment member 44′ and the tube 54 may be a single piece.

Still referring to FIG. 5A, the needle 18 is shown positioned entirely within the lumen 30, with the end 16 of the catheter 12 distal of the needle tip 20. Structurally, the needle 18 may be mounted to a needle hub (not shown) for movement with respect to the lumen 30 of the catheter 12.

Referring now to FIG. 5B, the abutment member 44″ is shown in its second configuration. Specifically, as shown, the distal portion 48 of the abutment member 44″ is shown extended from the distal end 16 of the catheter 12. Due to its preformed pancake shape, the distal portion 48 of the pancake shaped abutment member 44″ flares radially when inflated beyond the distal end 16 of the catheter 12. As shown in FIG. 5B, the needle 18 is still retracted within the catheter 12.

Referring now to FIG. 5C, it can be seen that the needle 18 has been advanced to extend the needle tip 20 beyond the distal end 16 of the catheter 12 and the abutment member 44″. Preferably, the needle tip 20 extends between 3 and 7 millimeters beyond the surface 50 of the abutment member 44″.

Referring now to FIGS. 5A-5C collectively, the operation of the present invention may be understood. Initially, the injector 10 is configured as in FIG. 5A with the abutment member 44′ in its first configuration. When an intra myocardial injection is desired, the injector 10 is placed adjacent myocardial tissue 42. Next, as shown in FIG. 5B, fluid such as iodinated saline is transferred from the reservoir 11 to the abutment member 44 and the distal portion 48 of the abutment member 44 expands in the distal direction. The abutment member 44 expands until it reaches its second configuration. In the second configuration, the distal portion 48 of the abutment member 44″ flares radially and provides a barrier between the distal end 16 of the catheter 12 and the myocardial tissue 42. When the injector 10 is moved toward the myocardial tissue 42, the surface 50 of the abutment member 44 abuts the tissue 42 and prevents further movement of the catheter 12 in the distal direction. Further, the surface 50 may contact and engage the myocardial tissue 42 to anchor the injector 10 in position.

With the abutment member 44″ in its second configuration, the needle 18 may be advanced to extend the needle tip 20 as shown in FIG. 5C. Advancement of the needle 18 may be accomplished by pushing the needle hub (not shown) relative to the catheter 12, or otherwise pushing the needle 18 relative to the catheter 12 as known in the art. As a result of its advancement, the needle tip 20 extends beyond the distal end 16 of the catheter 12 and beyond the surface 50 of the abutment member 44″ to penetrate the myocardial tissue 42 to an approximate depth of between 3 and 7 millimeters. When the myocardial tissue 42 has been penetrated by the needle tip 20 at the appropriate depth, a medicament or other treatment fluid may be injected through the needle tip 20 as is well known in the art.

After the injection has been performed, the needle 18 is retracted within the lumen 30, as illustrated in FIG. 5B. Then the abutment member 44′ is deflated by transferring fluid from the abutment member 44 to the fluid reservoir 11, as illustrated in FIG. 5A. Thereafter, the injector 10 is removed from the patient's vasculature.

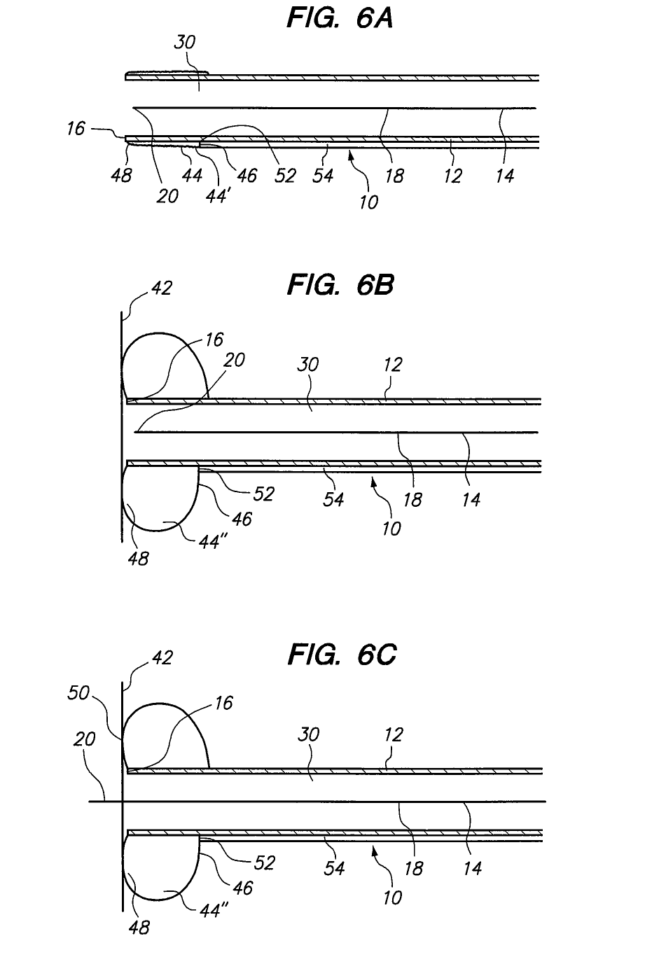

Turning now to FIG. 6A, the internal features of another embodiment of the injector 10 may be understood. As shown in FIG. 6A, the catheter 12 forms a lumen 30 that extends along the axis 14 to the distal end 16. Unlike in FIG. 4, the abutment member 44′ is deflated and positioned substantially alongside the catheter 12, i.e., in its first configuration. As shown, when in its first configuration, the abutment member 44′ is substantially tube-like. Specifically, the proximal portion 46 and the distal portion 48 of the abutment member 44′ are substantially cylindrical. For purposes of the present invention, the proximal portion 46 of the abutment member 44 may be mounted to the catheter 12.

In FIG. 6A, it is further shown that the abutment member 44 includes a port 52 that is in fluid communication with a tube 54. For purposes of the present invention, the tube 54 is connected to the fluid reservoir 11 (shown in FIG. 1) to transfer fluid between the reservoir 11 and the abutment member 44′ to cause movement of the abutment member 44′. In certain embodiments, the abutment member 44′ and the tube 54 may be a single piece. Further, while the tube 54 is positioned within the lumen 30 in FIG. 6A, it may be positioned outside of the catheter 12 as shown in FIG. 5A.

Still referring to FIG. 6A, the needle 18 is shown positioned entirely within the lumen 30, with the end 16 of the catheter 12 distal of the needle tip 20. Structurally, the needle 18 may be mounted to a needle hub (not shown) for movement with respect to the lumen 30 of the catheter 12.

Referring now to FIG. 6B, the abutment member 44″ is shown in its second configuration. Specifically, as shown, the distal portion 48 of the abutment member 44″ is shown extended from the distal end 16 of the catheter 12. Due to its preformed shape, the abutment member 44″ flares radially when inflated beyond the distal end 16 of the catheter 12. As shown in FIG. 6B, the needle 18 is still retracted within the catheter 12.

Referring now to FIG. 6C, it can be seen that the needle 18 has been advanced to extend the needle tip 20 beyond the distal end 16 of the catheter 12 and the abutment member 44″. Preferably, the needle tip 20 extends between 3 and 7 millimeters beyond the surface 50 of the abutment member 44″.

Referring now to FIGS. 6A-6C collectively, the operation of the present invention may be understood. Initially, the injector 10 is configured as in FIG. 6A with the abutment member 44′ in its first configuration. When an intra myocardial injection is desired, the injector 10 is placed adjacent myocardial tissue 42. Next, as shown in FIG. 6B, fluid is transferred from the reservoir 11 to the abutment member 44 and the distal portion 48 of the abutment member 44 expands radially outward and in the distal direction. The abutment member 44 expands until it reaches its second configuration. In the second configuration, the distal portion 48 of the abutment member 44″ provides a barrier between the distal end 16 of the catheter 12 and the myocardial tissue 42. When the injector 10 is moved toward the myocardial tissue 42, the surface 50 of the abutment member 44 abuts the tissue 42 and prevents further movement of the catheter 12 in the distal direction. Further, the surface 50 may contact and engage the myocardial tissue 42 to anchor the injector 10 in position.

With the abutment member 44″ in its second configuration, the needle 18 may be advanced to extend the needle tip 20 as shown in FIG. 6C. Advancement of the needle 18 may be accomplished by pushing the needle hub (not shown) relative to the catheter 12, or otherwise pushing the needle 18 relative to the catheter 12 as known in the art. As a result of its advancement, the needle tip 20 extends beyond the distal end 16 of the catheter 12 and beyond the surface 50 of the abutment member 44″ to penetrate the myocardial tissue 42 to an approximate depth of between 3 and 7 millimeters. When the myocardial tissue 42 has been penetrated by the needle tip 20 at the appropriate depth, a medicament or other treatment fluid may be injected through the needle tip 20 as is well known in the art.

After the injection has been performed, the needle 18 is retracted within the lumen 30, as illustrated in FIG. 6B. Then the abutment member 44″ is deflated by transferring fluid from the abutment member 44 to the fluid reservoir 11, as illustrated in FIG. 6A. Thereafter, the injector 10 is removed from the patient's vasculature.

While the particular Myocardial Injector With Balloon Abutment as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. An intra myocardial injector which comprises:
a catheter having a distal end, said catheter being formed with a lumen defining a longitudinal axis;
a needle having a tip, said needle being mounted within the lumen of the catheter for axial movement therein;
an inflatable abutment member, said abutment member having a first configuration, wherein the abutment member is substantially deflated, and a second configuration, wherein the abutment member is substantially inflated and extends radially from the distal end of the catheter;
a means for selectively inflating and deflating the abutment member to move the abutment member between the first configuration and the second configuration; and
a means for advancing the needle tip through the distal end of the catheter and beyond the abutment member to penetrate myocardial tissue to perform an injection, when said abutment member is in its second configuration to prevent contact between said catheter and said myocardial tissue during the injection.

2. An injector as recited in claim 1 wherein the abutment member includes a distal portion that extends radially in the second configuration.

3. An injector as recited in claim 2 wherein the abutment member is comprised of a flexible bladder.

4. An injector as recited in claim 3 wherein the abutment member includes a port in fluid communication with the inflating and deflating means.

5. An injector as recited in claim 4 wherein saline is used to inflate and deflate the abutment member.

6. An injector as recited in claim 5 further comprising a saline reservoir in fluid communication with the port in the abutment member.

7. An injector as recited in claim 1 wherein the abutment member is substantially tube-like in the first configuration, and is substantially radially flared in the second configuration.

8. An intra myocardial injector which comprises:
a catheter having a distal end, said catheter being formed with a lumen defining a longitudinal axis;
a needle having a tip, said needle being mounted within the lumen of the catheter for axial movement therein;
an inflatable abutment member having a port, said abutment member having a first configuration, wherein the abutment member is substantially deflated, and a second configuration, wherein the abutment member is substantially inflated and extends radially from the distal end of the catheter;
a fluid reservoir in fluid communication with the port of the abutment member;
a means for selectively transferring fluid between the fluid reservoir and the abutment member to move the abutment member between the first configuration and the second configuration; and
a means for advancing the needle tip through the distal end of the catheter and beyond the abutment member to penetrate myocardial tissue to perform an injection, when said abutment member is in the second configuration to prevent contact between said catheter and said myocardial tissue during the injection.

9. An injector as recited in claim 8 wherein the abutment member includes a distal portion that extends radially in the second configuration.

10. An injector as recited in claim 9 wherein the abutment member is comprised of a flexible bladder.

11. An injector as recited in claim 10 wherein the abutment member includes a central void that the needle tip passes through when the abutment member is in the second configuration.

12. An injector as recited in claim 8 wherein the fluid is saline.

13. An injector as recited in claim 8 wherein the abutment member is substantially tube-like in the first configuration, and is substantially radially flared in the second configuration.

14. A method for performing an intra myocardial injection which comprises the steps of:
providing an injector comprising a catheter having a distal end, said catheter being formed with a lumen defining a longitudinal axis; a needle having a tip, said needle being mounted within the lumen of the catheter for axial movement therein; and an inflatable abutment member, said abutment member having a first configuration, wherein the abutment member is substantially deflated, and a second configuration, wherein the abutment member is substantially inflated and extends radially from the distal end of the catheter;
positioning the injector at a desired position adjacent myocardial tissue;
transferring fluid to the abutment member to move the abutment member from the first configuration to the second configuration to prevent contact between said catheter and said myocardial tissue; and
advancing the needle tip through the distal end of the catheter and beyond the abutment member to penetrate myocardial tissue to perform the injection.

15. A method as recited in claim 14 wherein the abutment member includes a distal portion that extends radially in the second configuration.

16. A method as recited in claim 15 wherein the abutment member is comprised of a flexible bladder.

17. A method as recited in claim 16 wherein the abutment member includes a port in fluid communication with a fluid source, and wherein the transferring step includes pumping fluid through the port to inflate the abutment member.

18. A method as recited in claim 17 wherein the fluid is saline.

19. A method as recited in claim 18 wherein the fluid source is a saline reservoir.

20. A method as recited in claim 14 wherein the abutment member is substantially tube-like in the first configuration, and is substantially radially flared in the second configuration.
